(19) United States
(12) Patent Application Publication
(10) Pub. No.: US 2015/0140352 A1
(43) Pub. Date: May 21, 2015

(54) SEMIFINISHED PRODUCT AND HIGH-STRENGTH DEGRADABLE IMPLANT FORMED THEREFROM

(71) Applicant: BIOTRONIK AG, Buelach (CH)

(72) Inventors: Ullrich Bayer, Bad Doberan (DE); Okechukwu Anopuo, Rostock (DE); Bernd Block, Ostseebad Nienhagen (DE)

(21) Appl. No.: 14/525,841

(22) Filed: Oct. 28, 2014

Related U.S. Application Data
(60) Provisional application No. 61/905,299, filed on Nov. 18, 2013.

(51) Int. Cl. A61L 31/02; C22C 23/02; B21C 23/00; C22C 23/06

(52) U.S. Cl. CPC A61L 31/022; C22C 23/06; C22C 23/02; B21C 23/001
USPC 428/600; 420/406; 420/409; 420/410; 72/271; 428/544

(57) ABSTRACT

A semifinished product for an implant and implants produced from the semifinished product, the semifinished product comprising or consisting of a region of a magnesium alloy, which is characterized by a grain size gradient of the magnesium alloy between two opposed surfaces from ≦3 μm to ≧8 μm, in each case in relation to the average grain size. Use of the semifinished product for producing corresponding implants, and also a method for producing semifinished products.

### CROSS REFERENCE TO RELATED APPLICATIONS

This invention claims benefit of priority to U.S. provisional patent application Ser. No. 61/905,299, filed Nov. 18, 2013; the content of which is herein incorporated by reference in its entirety.

### TECHNICAL FIELD

The invention relates to a semifinished product for an implant, comprising or consisting of a region of a magnesium alloy, which is characterized by a grain size gradient of the magnesium alloy between two opposite surfaces from ≦3 μm to ≧8 μm, in each case in relation to the average grain size. The invention also relates to implants produced from this semifinished product. In addition, the invention relates to the use of the semifinished product according to the invention for producing corresponding implants and also to a method for producing semifinished products according to the invention.

### BACKGROUND

In particular for orthopedic implants, there is a constant need for implant materials that meet high demands in terms of mechanical loading, such as tensile strength, bending strength and compressive strength, and are suitable for uses where high surface pressures occur. Examples of such orthopedic implants are cannulated bone screws, Kirschner wires, and implants for spine surgery such as vertebral body stenting (VBS).

It is desirable from a multiplicity of viewpoints for the implants to be able to consist of magnesium alloys, since these alloys demonstrate particularly good processability and/or compatibility properties in a large number of fields of use. In particular, it is preferable if the alloy is bioresorbable, such that explantation of the implant once it has performed its function is unnecessary.

In many fields, in particular in those with high mechanical demands on the implant, it was not previously possible to use magnesium alloys. In the prior art, high-strength iron-based alloys alloyed with Mn, Pd and/or Pt in order to increase the degradation rate were used instead, for example. Besides long degradation times, these alloys have the further disadvantage that stress-shielding effects occur due to the high moduli of elasticity inherent to these alloys, and the actual function of the implant (the biological anchoring to bone tissue) is thus counteracted in a lasting manner.

An alternative material in the prior art is constituted by metal glasses based on Mg, Zn, Ca, which, inter alia, have the disadvantage of excessively low formability and excessively low range of variation of producible semifinished products.

In particular from the viewpoint of biodegradability, (non-extruded) medium-strength degradable magnesium alloys, such as WE 43, are also used in the prior art, but either do not have the strength necessary for the introduction of threads for example, or have an excessively high degradation rate due to high grain size.

Metal-cement composites, such as iron/brushite foams, are known in the prior art as further alternative material compositions, but have the disadvantage inter alia of particle generation during implantation, thus increasing the risk of subsequent local inflammation.

### SUMMARY OF THE INVENTION

The object of the present invention is to provide an improved semifinished product, based on magnesium alloys, for the production of implants. In particular, it is of interest to use alloys that also meet increased mechanical demands and are preferably also biodegradable (bioresorbable). In particular, it is a preferred objective to widen the field of use of degradable implants to include areas that previously were accessible only to high-strength, non-degradable materials, such as titanium, L605 and 316L.

The object forming the basis of the invention is achieved by a semifinished product for an implant, comprising or consisting of a region formed from an Mg alloy, which is characterized by a grain size gradient of the Mg alloy between two opposite surfaces from ≦3 μm to ≧8 μm, preferably from ≦2 μm to ≧10 μm, in each case in relation to the average grain size.

### BRIEF DESCRIPTION OF THE DRAWINGS

Embodiments of the invention can be better understood with reference to the following drawings, which form part of the specification.

FIGS. 1A-C depict a mold of a blank to be formed in the shape of a hollow cylinder.

FIG. 2 depicts a schematic of a half section of an outer part of a forming die used having a sharp radius of curvature of 0.01 mm.

FIG. 3 depicts a schematic of a half section of an outer part of a forming die used having a large radius of curvature of 5.0 mm.

### DETAILED DESCRIPTION OF PREFERRED EMBODIMENTS

In a first aspect of the invention a semifinished product for an implant is provided, which comprises or consists of a region formed from an Mg alloy, which is characterized by a grain size gradient of the Mg alloy between two opposite surfaces from ≦3 μm to ≧8 μm, preferably from ≦2 μm to ≧10 μm, in each case in relation to the average grain size.

Within the meaning of this text, a magnesium alloy is an alloy which comprises at least one proportion of ≧80% by weight of magnesium.

Further preferred constituents of magnesium alloys to be used in accordance with the invention are Zn, Ca, Li, Mn and rare earths, preferably in the following proportions (in each case in % by weight in relation to the total alloy):

### Li: 0.01-3.0

### Ca: 0.01-2.0

### Zn: 0.01-5.0

### Mn: 0.01-0.5

### Nd: 0.01-1.5

### Dy: 0.01-1.0

Gd: 0.01-1.0 and/or

### Eu: 0.01-0.5

Preferred magnesium alloys for the semifinished product according to the invention are:

1.) WE 43 (3.7 to 4.3% by weight Y; 2.4-4.4% by weight of rare earths; 0.4-0.7% by weight of Zr; the rest being formed by magnesium)
2.) AZ 31 (2.5-3.5% by weight of Al; 0.7-1.3% by weight of Zn; 0.2-0.4% by weight of Mn; in each case at most 0.05% by weight of Si and Cu; at most 0.04% by weight of Ca; in each case at most 0.005% by weight of Fe and Ni; the rest being formed by magnesium)
3.) AM 60 (5.0-6.0% by weight of Al; 1.5-2.5% by weight of Ca; 0.2-0.5% by weight of Mn; the rest being formed by magnesium).

The grain size characteristic G is determined from the average number of grains m, which are counted on a square millimeter cross-sectional area of the sample. The grain size determination is carried out using a graded image charts in accordance with ASTM E 112-12.

Here, the grain size is established by means of light microscopy with the aid of standardized image charts. Each of these template-like image charts illustrates an average grain size. These image charts are compared with the grain structure of the etched metallographic ground section with 100 times magnification. The grain size is read off from the image chart showing the greatest possible conformity with the ground section.

Due to the gradient, provided in accordance with the invention, of the grain size of the magnesium alloy to be used in accordance with the invention, the semifinished product comprises regions having different mechanical properties, which are determined substantially by the grain size in the alloy. Here, it has surprisingly been found that the semifinished products according to the invention and implants produced therefrom have a good property combination of yield point values and tensile strength values with sufficient elongation at failure. Here, the regions with the larger average grain size are suitable for use in fields in which the (later) implants are particularly stressed mechanically and/or corrosively.

This makes it possible to use the semifinished products according to the invention for implants for application in orthopedics, in which high demands are placed on tensile strength, bending strength and compressive strength and high surface pressures are also present.

Examples for this include cannulated bone screws, Kirschner wires, and implants for spine surgery, such as vertebral body stenting (VBS). The implants developed from the semifinished product material according to the invention can be used both in vertebral surgery and in bone fixing and stabilization, with or without substances accelerating osteosynthesis.

In principle, the semifinished products according to the invention or the implants produced therefrom may also be equipped with substances which assist the function of the implant.

For example, these may be compounds containing calcium phosphate, such as hydroxylapatite (HAP) or bone morphogenetic proteins (BMPs), with which implants of this type are surface-functionalized in respect of accelerated osteosynthesis.

These may also be surface-active phosphonates. Copolymers, such as 4-vinylpyridene with vinylbenzyl phosphonate or dimethyl-(2-methacryloyloxy) ethyl phosphonate thus positively influence osseointegration and also prevent the adhesion of bacteria to the implant surfaces. In terms of an alloy, the elements Ca and Zn in particular are conceivable and, with progressive degradation of the resorbable magnesium implant, promote integration into the bone substance.

In accordance with the invention, it is preferable for the semifinished product according to the invention to constitute a planar structure or a hollow profile.

A planar structure within the meaning of this text is a body which has two primary surfaces (front side and rear side) of approximately equal size, wherein these two primary surfaces are arranged opposite one another and the body extends substantially in only two spatial directions.

Within the meaning of this text, a hollow profile is a body which comprises an enclosed hollow space (for example a hollow sphere) or an open hollow space (for example a tube).

Preferred hollow profiles within the meaning of this text are open cylinders.

In accordance with the invention, with the preferred semifinished products according to the invention, which constitute a planar structure, the grain size gradient runs from one primary surface of the planar structure to the other. In the case of hollow profiles, the grain size gradient runs in accordance with the invention from the inner surface to the outer surface of the body.

Surfaces which have different mechanical properties are thus produced, but cooperate positively for the semifinished product and the implant manufactured therefrom. In addition, it is possible to utilize properties of one of these surfaces for specific purposes (see further below).

In accordance with the invention, it is preferable if the magnesium alloy is bioresorbable.

Within the meaning of the present invention, bioresorbable means that, with incorporation into the human or animal body, preferably into the human body, the alloy is degraded at a rate of 1 μm to 3 μm/week. This means that an implant having a wall thickness of 150 μm at a degradation rate of 1 μm/week is fully degraded in 75 weeks. With a degradation rate of 3 μm/week, this implant would be fully degraded after just 25 weeks.

Strength properties of non-degradable alloys, such as titanium and stainless steel 316L, can surprisingly be achieved to a sufficient extent with the bioresorbable semifinished products according to the invention. This was not to be expected as a matter of course due to the physical conditions, such as lattice structure and atomic binding forces, predefined by the primary alloy element Mg. With the bioresorbable semifinished products now provided in accordance with the invention, implants can be produced that, due to their mechanical properties, are equipped such that clinicians familiar with the implantation of non-degradable orthopedic implants do not have to make any significant changes to the way in which surgery is performed. This also includes the usability of the clinical surgical instruments usually provided, such as screwdrivers, torque wrenches and pliers.

Here, a semifinished product according to the invention that is produced in an extrusion method is preferred.

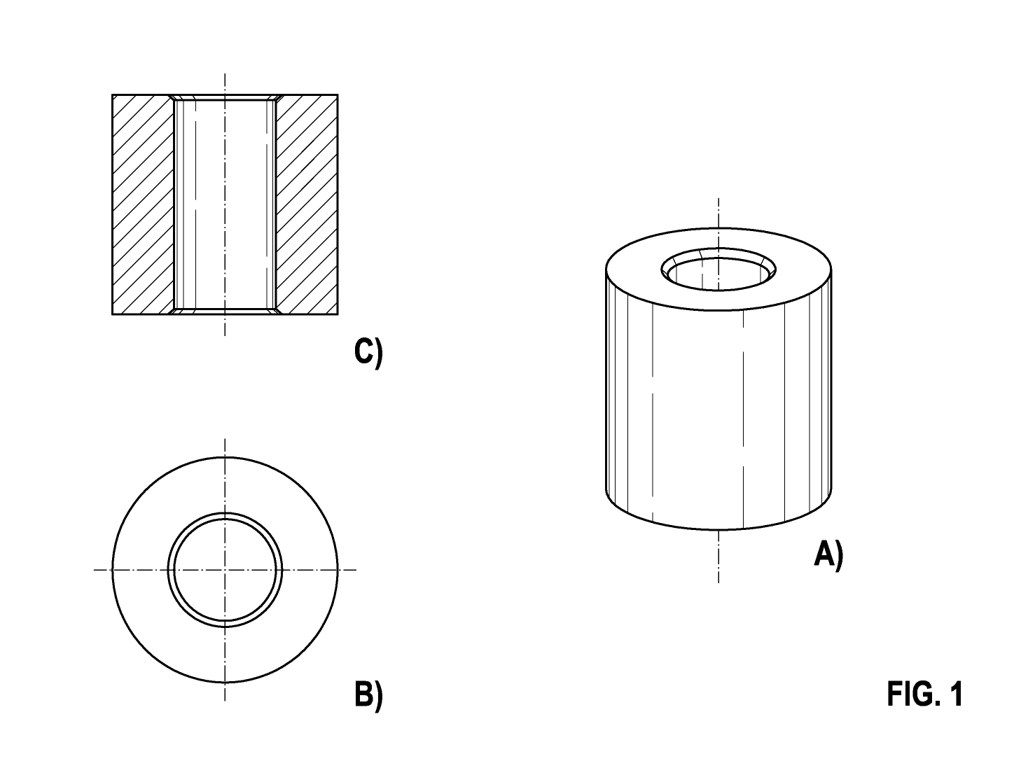

The semifinished products according to the invention can be produced particularly favorably and effectively via an appropriately designed extrusion method. Suitable preferred measures in this context are as follows:
a specific mold of the blank to be formed in the shape of a hollow cylinder, a preferred variant is illustrated in FIGS. 1A to C.
a suitable die geometry for the extrusion die, with which specific forming grades can be achieved over the cross section of the semifinished product which in turn generate gradients in the grain size between the inner and outer face. Here, the following measures in particular are to be mentioned.
In particular, it is a question of producing differently shaped edges in the interior of the die. In the case of sharp edges, that is to say edges with a small radius of curvature (for example R=0.01 mm), the blank is formed more than in the case of an edge with a large radius of curvature (for example R=5.0 mm) In other words, when the magnesium (or the magnesium alloy) flows over rather “angular” structures, the originally provided microstructure is influenced (shattered) to a greater extent than in the case of forming around die structures that are more round. The grains are less compacted at these points, and the originally provided dislocation density is less increased compared to forming around sharp edges, and the originally provided average grain size remains relatively unchanged.
a specific forming rate between 0.01 s⁻¹ and 35 s⁻¹.
   the omission of a subsequent heat treatment or a specific heat treatment after the extrusion process in order to stabilize the microstructure gradient over the cross section of the extruded product. Preferred parameters for this heat treatment are 200° C. to 500° C. for a period of time from 3 to 180 minutes.

A microstructure can thus be achieved which is characterized by extremely fine grain sizes (average grain diameter ≦3 μm (preferably ≦2 μm) on one side of the semifinished product cross section and grain sizes ≧8 μm (preferably ≧10 μm, particularly preferably ≧15 μm) on the opposite semifinished product side.

Here, the two semifinished product sides (top and bottom or inside and outside) can be swapped by the respective arrangement of the forming dies.

A semifinished product according to the invention wherein the semifinished product is a hollow profile in the shape of a cylinder and the outer face of the cylinder lateral surface comprises or consists of the alloy having the larger average grain size, and the inner face of the cylinder lateral surface comprises or consists of the alloy having the smaller average grain size is preferred accordingly.

A semifinished product according to the invention may also be preferred for other purposes, wherein the semifinished product is a hollow profile in the form of a cylinder and the outer face of the cylinder lateral surface comprises or consists of the alloy having the smaller average grain size and the inner face of the cylinder lateral surface comprises or consists of the alloy having the larger average grain size.

The extrusion process is preferably carried out with a previously solution-annealed material. In this state, the structure has large grains and no sediments. This means that all alloy elements are dissolved in the matrix. The subsequent extrusion process is carried out at temperatures and forming pressures which on the one hand eliminate renewed sediment formation and on the other hand lead to submicroscopic grain sizes. Here, the following is of particular importance:
the fact that the temperature falls below the temperature at the start of dynamic recrystallisation,
the fact that areas of different deformation grades are produced over the component cross section due to a specific die geometry, and/or
the fact that the subsequent recrystallization annealing is managed in terms of time and temperature such that the zones having different deformation grades recrystallize such that the respective different forming grade is maintained.

This results in a different distribution of grain sizes over the component cross section and also in a homogenous distribution of grain formation.

Different magnesium alloys can be set to the desired grain size gradient using the suitable extrusion die.

The grain size gradient, as described above, means that surprisingly good mechanical properties can be achieved for the semifinished products according to the invention.

Accordingly, a semifinished product wherein said semifinished product in the region of the magnesium alloy has a tensile strength of ≧400 MPa, preferably ≧440 MPa, and/or a yield point of ≧225 MPa, preferably ≧250 MPa, and/or an elongation at failure of ≧4%, preferably ≧4.5%, is preferably used in accordance with the invention.

These preferred mechanical properties make the semifinished products according to the invention or the implants produced therefrom, even in their preferred bioresorbable form, accessible for a large number of applications which previously were closed in particular to bioresorbable magnesium alloys.

It is preferable in this context for the semifinished products according to the invention to be produced with an end contour close to that of the implant to be produced therefrom, such that only little manufacturing outlay is necessary to produce the actual implant.

In accordance with the invention, a semifinished product wherein the yield point of the region of the magnesium alloy having the smaller average grain size is ≧40 MPa, preferably ≧45 MPa, greater than the yield point of the region of the Mg alloy having the larger average grain size is preferred.

Due to these relatively large differences in the yield points, embodiments that make a subsequent hot forming of this region necessary or that are at least favored by such an operation can be implemented in the region of the magnesium alloy having the larger average grain size. For example, the provision of a thread, in particular of an outer thread, without impairing the mechanical properties too significantly, is thus only possible by the embodiment according to the invention of the semifinished product according to the invention.

For example, it is thus possible to produce screws which enable a torque which is increased by 30% when screwing into the bone compared to bone screws of which the threads have been produced by means of machining and from the same alloy material without the specific embodiment of the semifinished product according to the invention (grain size gradient).

The invention also relates to an implant produced or producible from a semifinished product according to the invention. This implant has the above-described advantages, in particular in its preferred form the property of bioresorbability, such that it no longer has to be explanted once its tasks have been performed.

An implant according to the invention that, in the region of the magnesium alloy having the larger average grain size, has a thread or another topography enlarging the actual surface is preferable. This is particularly advantageous where a large surface is desired for more intensive surface contact with the surrounding bone. The incorporation rate of the implant into the bone is thus increased, and the bone can be mechanically stressed already at an earlier moment in time.

An implant according to the invention selected from the group consisting of internally cannulated bone screws, pico-pin screws, bone clamps, intramedullary nails, intramedullary rods, Cerclage wire, and wire cages bracing the spine, is preferred. The invention also relates to the use of a semifinished product according to the invention for producing an implant.

The invention further relates to a method for producing a semifinished product according to the invention, comprising the following steps
a) providing an Mg alloy,
b) providing an extrusion device for the alloy, which is designed such that, during the extrusion process, two opposed surfaces formed from alloy constituents are produced that have been subjected to a different extrusion rate (and therefore a different degree of deformation), and
c) extruding the alloy by means of the device.

Due to a specific arrangement, characteristic for the invention, of inner and outer dies (or in the region for the upper face and lower face of the extruded object), the different grain sizes over the component cross section are produced.

Preferred semifinished products produced by methods according to the invention are in this case rotationally symmetrical hollow profiles.

The invention also relates to the production of an implant according to the invention formed from a semifinished product according to the invention.

Generally, it can be noted that the stressing occurring in medical applications, in particular for the preferred implants, requires mechanical properties that could previously only be achieved by use of permanent, that is to say non-degradable (non-bioresorbable) metals, such as CoCr, stainless steel 316L, and titanium. In particular, the surface pressures, which occur in the thread of securely tightened bone screws, and also the bending stiffness of bone clamps make the use of higher-strength materials necessary. The preferred (bioresorbable) implant according to the invention can continue its degradation process (bioresorbtion), after the process of bone healing, without negative consequences for the body. A further operation in order to remove the implant is not necessary.

Furthermore, a general advantage of the implant to be used in accordance with the invention is the fact that magnesium alloys have only a relatively low modulus of elasticity, for example of 44 GPa. This prevents stress-shielding effects, which, in conventional implant materials having a high modulus of elasticity, lead to negative interaction with the bone surface. Furthermore, the semifinished products according to the invention and the implants produced therefrom enable use in applications in which the resorbability of the implant is important and could not previously be covered only by degradable polymers.

In addition, hollow wires and hollow rods as implants according to the invention offer the possibility of internal filling, for example with substances accelerating osteosynthesis, for example preferably hydroxylapatite and/or BMPs (bone morphogenetic proteins).

### EXAMPLES

### Used Die and Special Instructions

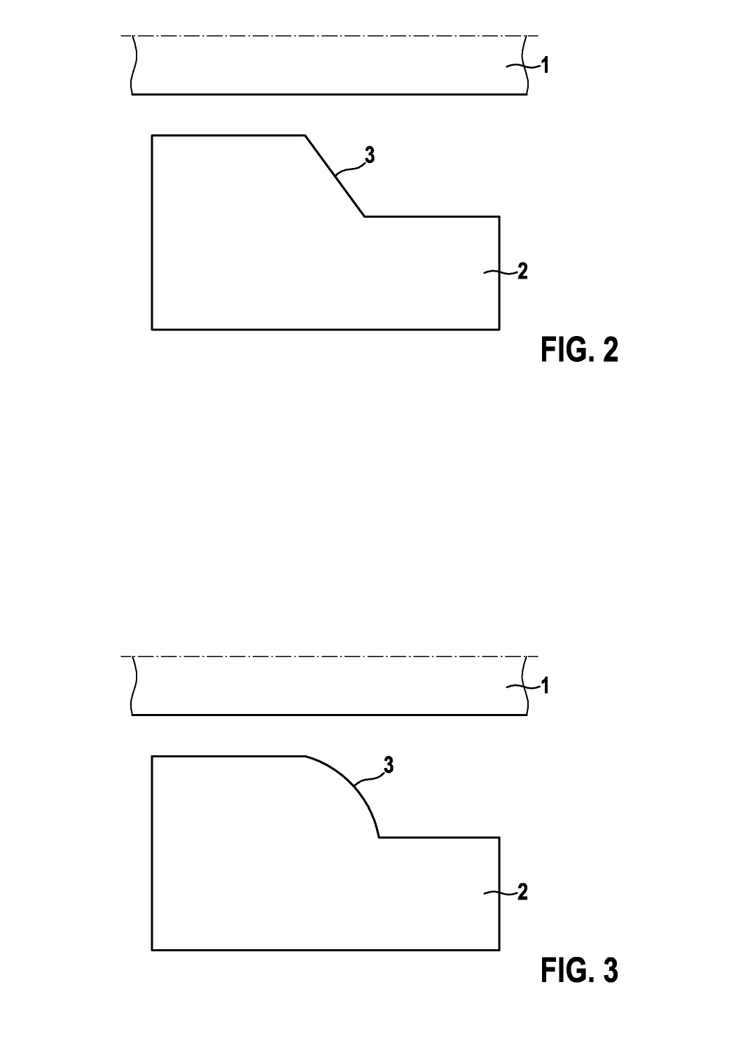

FIGS. 2 and 3 show schematic half-sections of outer parts of the forming die used. In FIG. 2, the sharp radius of curvature of 0.01 mm is shown, and in FIG. 3, the large radius of curvature of 5.0 mm is illustrated. With use of a die according to FIG. 3 for the extrusion process, the magnesium flowing directly past the sharp edge 3 of the die 2 experiences a higher deformation than the material volume located further toward the die insert 1. The above-mentioned effects in terms of a grain size gradient are thus produced. The average grain size in this example therefore increases from the outer face to the inner face of the extruded tube.

With use of the die illustrated in FIG. 3, the opposite effect is produced. At the large radius of curvature 3 of the die 2, the magnesium is able to flow past without considerable deformation. The originally provided grain size is maintained practically fully in this region close to the outer face of the tube. The volumes located further inwardly toward the middle of the tube are deformed to a greater extent by the pressing effect of the die insert 1. A gradient in the average grain size from the inside out thus results. This leads to the relatively large grains, described in this text, in the region of the tube outer wall. These in turn allow a subsequent further deformation of the outer surface of the tube (for example by means of thread rolling). This variant is therefore used in the production of internally cannulated bone screws.

The first-mentioned variant (with the smaller grains to the outside) is used in products which make necessary a hard outer surface. For example, these are bone pins, wire cages for the spinal column, or Cerclage wire, of which the primary loading in the event of implantation occurs on the outer surfaces.

### Example 1

Starting material: Mg alloy WE 43

Hollow forward extrusion of a sleeve at an extrusion temperature of 350° C., an extrusion rate of 5 mm/min, without subsequent heat treatment

The tube produced has the following dimensions:

length (L)=60 mm
outer diameter (OD)=3.1 mm
inner diameter (ID)=1.5 mm
wall thickness (WT)=0.8 mm

From the tube thus processed, internally cannulated cortical screws and/or internally cannulated spongiosa screws are produced by means of subsequent machining or non-cutting processing of the outer surface. The thread outer diameter is 3.0 mm here, and the diameter of the inner bore is 1.5 mm. A fixation of fragments of a bone fracture and subsequent bone healing or reintervention is thus possible.

### Example 2

Starting material: Mg alloy WE 43

Hollow forward extrusion of a sleeve at an extrusion temperature of 350° C., an extrusion rate of 5 mm/min, without subsequent heat treatment

The tube produced has the following dimensions:

length (L)=60 mm
outer diameter (OD)=1.8 mm
inner diameter (ID)=0.8 mm
wall thickness (WT)=0.5 mm

From the tube thus processed, internally cannulated cortical screws and/or internally cannulated spongiosa screws for small fragments in the finger area of children are produced by means of subsequent machining or non-cutting processing of the outer surface. The screws have a length between 5 and 20 mm. Here, the thread outer diameter is 1.5 mm, and the diameter of the inner bore is 0.8 mm. A fixation of fragments of a broken finger of a growing child is thus possible. Due to the degradation of the material, the bone is not hindered from growing together. In this case too, there is no need for a further operation.

### Example 3

Starting material: Mg alloy WE 43

Hollow forward extrusion of a sleeve at an extrusion temperature of 350° C., an extrusion rate of 5 mm/min, without subsequent heat treatment

The tube produced has the following dimensions:

length (L)=170 mm
outer diameter (OD)=2.2 mm
inner diameter (ID)=0.8 mm
wall thickness (WT)=0.7 mm

From the tube thus processed, internally cannulated Kirschner wires are manufactured. These are then provided with transverse bores (D=0.3 mm) at a distance in each case of 5 mm. Once the wire is finished, the inner diameter is filled with a substance accelerating osteosynthesis, such as nanocrystalline hydroxylapatite powder. Once the Kirschner wire has been introduced into the spongiosa of the thigh bone, the degradation of the Mg alloy begins. The hydroxylapatite powder, which is also released with continued degradation of the Kirschner wire, accelerates osteosynthesis.

### Example 4

Starting material: Mg-alloy containing 5.0% Zn and 0.25% Ca

Hollow forward extrusion of a sleeve at an extrusion temperature of 200° C., an extrusion rate of 3 mm/min, without subsequent heat treatment

The tube thus manufactured in the tension test demonstrates the following mechanical properties:

### Rₘ=445 MPa; R_{p0,2}=267 MPa; Aₜ=5.0%

the tube has the following dimensions:
length (L)=170 mm
outer diameter (OD)=2.0 mm
inner diameter (ID)=0.7 mm
wall thickness (WT)=0.65 mm

Due to the setting of different degrees of forming in the regions close to the inner surface and outer surface, the tube has different grain sizes. In this case, the regions close to the outer surface have an average grain size of >15 μm. By contrast, the regions close to the inner surface have average grain sizes of <2 μm. A gradient in the mechanical properties is thus produced. These properties have the following effects:

The yield point in the regions close to the outer surface is approximately 50 MPa lower than the integral yield point, measured over the entire component cross section. Subsequent hot forming of the outer surface is thus possible. This is applied in the hot rolling of an outer thread, which is only possible by this process chain. Here, the sleeve is slid onto a die insert and the outer thread is rolled in, in the temperature range between 190° C. and 240° C. The spongiosa screw produced can be screwed into the bone with a torqued that is increased by 30% compared to bone screws of which the threads have been manufactured from the same alloy by means of machining and without the preceding process chain.

Further embodiments of this example are internally cannulated cortical screws and internally cannulated spongiosa screws for small fragments in the finger area of children. The screws have a length between 6 and 20 mm. The thread outer diameter is 1.8 mm here, and the diameter of the inner bore is 1.2 mm. The thread depth is 0.3 mm. A fixation of fragments of a broken finger of a growing child is thus possible. Due to the degradation of the material, the bone is not hindered from growing together. In this case too, there is no need for a further operation.

It will be apparent to those skilled in the art that numerous modifications and variations of the described examples and embodiments are possible in light of the above teaching. The disclosed examples and embodiments are presented for purposes of illustration only. Other alternate embodiments may include some or all of the features disclosed herein. Therefore, it is the intent to cover all such modifications and alternate embodiments as may come within the true scope of this invention.

What is claimed is:

1. A semifinished product for an implant, comprising a region formed from an Mg alloy, which is characterized by a grain size gradient of the Mg alloy between two opposed surfaces from ≦3 μm to ≧8 μm, optionally from ≦2 μm to ≧10 μm, in each case in relation to the average grain size.

2. The semifinished product as claimed in claim 1, wherein the semifinished product is a planar structure or comprises a hollow profile.

3. The semifinished product as claimed in claim 1, wherein the Mg alloy is bioresorbable.

4. The semifinished product as claimed in claim 1, wherein the semifinished product is produced by an extrusion method.

5. The semifinished product as claimed in claim 1, wherein the semifinished product is the hollow profile in a form of a cylinder and the outer face of the cylinder lateral surface comprises the alloy having the larger average grain size, and the inner face of the cylinder lateral surface comprises the alloy having the smaller average grain size.

6. The semifinished product as claimed claim 1, wherein the semifinished product comprises the hollow profile in the form of a cylinder and an outer face of the cylinder lateral surface comprises the alloy having the smaller average grain size, and the inner face of the cylinder lateral surface comprises the alloy having the larger average grain size.

7. The semifinished product as claimed in claim 1, wherein the semifinished product, in the region of the Mg alloy, has a tensile strength of ≧400, optionally ≧440 MPa, and/or a yield point of ≧225, optionally ≧250 MPa.

8. The semifinished product as claimed in claim 1, wherein the semifinished product, in the region of the Mg alloy, has an elongation at failure ≧4% preferably ≧4.5%.

9. The semifinished product as claimed in claim 1, wherein the yield point of the region of the Mg alloy having the smaller average grain size is ≧40 MPa, optionally ≧45 MPa, greater than the yield point of the region of the Mg alloy having the larger average grain size.

10. An implant produced or producible from a semifinished product as claimed in claim 1.

11. The implant as claimed in claim 10, wherein the implant, in the region of the Mg alloy having the larger average grain size, has a thread or radially extending surface grooves for increasing the surface.

12. The implant as claimed in claim 10, wherein the implant is in a form selected from the group consisting of internally cannulated bone screws, pico-pin screws, bone clamps, intramedullary nails, intramedullary rods, Cerclage wire, and wire cages bracing the spinal column.

13. A method for producing a semifinished product as claimed in claim 1, comprising the following steps:
a) providing an Mg alloy;
b) providing an extrusion device for the alloy, which is designed such that, during the extrusion process, two opposed surfaces formed from alloy constituents are produced that have been subjected to a different extrusion rate; and
c) extruding the alloy by means of the device.

14. A method for producing an implant as claimed in claim 10, comprising the following steps:
a) providing a semifinished product as claimed in claim 1; and
b) producing the implant from the semifinished product.
